SEQUENCE LISTING (10 sequences)

SEQ ID NO: 1 - DNA, 1200 bp, rat bombesin receptor subtype-3
atgtctcaaaggcagcctcagtcacctaatcagactttaatttccattacaaatgacacagaaacatcaagctctgccgtctccaacgatactacacctaaaggatggaccggagacaactctccaggaatagaagcactgtgtgccatctatatcacttatgctgtgatcatttcagtgggcatcctcggaaatgctatcctcatcaaagtctttttcaagactaaatccatgcaaacagttccaaatattttcatcaccagcctggcttttggagatctgttactcctgctgacttgtgtgccagtggatgcaacccactacctggcagagggatggctgtttggaaaggtcggttgtaaagtgctttccttcatccggctcacttctgtcggtgtatcagtgttcacgctaacaattctcagcgctgacagatacaaagcagtcgtgaagccacttgaacgacagccctccaatgccattctgaagacctgtgccaaagctggtggcatctggatcatggctatgatatttgctctgccagaggctatattctcaaatgtatacactttccaagatcctaacagaaacgtaacatttgaatcctgtaactcctaccctatctctgagaggcttttgcaggaaatacattctctgttgtgtttcttggtgttctacattatcccgctctcgattatctctgtctattattctttgattgccaggactctttacaaaagcaccttgaacataccgactgaggaacaaagccatgcccgaaagcagattgaatcccggaagagaattgccaaaacggtactggtgctggtggctctgttcgcactctgctggttgccgaatcacctcctgtatctctatcactcattcacttatgaaagctacgcagaaccttctgatgtccctttcgttgtcaccattttctctcgggtgctggctttcagtaattcctgcgtgaacccctttgctctgtattggctgagcaagaccttccagaagcattttaaggctcagctctgctgcttcaaggcagagcagcctgagcctcctcttggtgacaccccccttaacaacctcactgtgatggggcgggttccagctactgggagtgcacacgtctctgaaattagcgtgaccctgtttagtggcagtactgccaagaaaggagaggacaaagtttag

SEQ ID NO: 2 - protein, 342 aa, rat bombesin receptor subtype-3
MSQRQPQSPNQTLISITNDTETSSSAVSNDTTPKGWTGDNSPGIEALCAIYITYAVIISVGILGNAILIKVFFKTKSMQTVPNIFITSLAFGDLLLLLTCVPVDATHYLAEGWLFGKVGCKVLSFIRLTSVGVSVFTLTILSADRYKAVVKPLERQPSNAILKTCAKAGGIWIMAMIFALPEAIFSNVYTFQDPNRNVTFESCNSYPISERLLQEIHSLLCFLVFYIIPLSIISVYYSLIARTLYKSTLNIPTEEQSHARKQIESRKRIAKTVLVLVALFALCWLPNHLLYLYHSFTYESYAEPSDVPFVVTIFSRVLAFSNSCVNPFALYWLSKTFQKHFK

SEQ ID NO: 3 - protein, 399 aa, mouse bombesin receptor subtype-3
MSQRQSQSPNQTLISITNDTETSSSVAPNDTTHKGWTGDNSPGIEALCAIYITYAGIISVGILGNAILIKVFFKTKSMQTVPNIFITSLAFGDLLLLLTCVPVDATHYLAEGWLFGKVGCKVLSFIRLTSVGVSVFTLTILSADRYKAVVKPLERQPPNAILKTCAKAGGIWIVSMIFALPEAIFSNVYTFQDPNRNVTFESCNSYPISERLLQEIHSLLCFLVFYIIPLSIISVYYSLIARTLYKSTLNIPTEEQSHARKQIESRKRIAKTVLVLVALFALCWLPNHLLYLYHSFTYESYANHSDVPFVIIIFSRVLAFSNSCVNPFALYWLSKTFQQHFKAQLCCLKAEQPEPPLGDIPLNNLTVMGRVPATGSAHVSEISVTLFSGSSAKKGEDKV

SEQ ID NO: 4 - protein, 399 aa, human bombesin receptor subtype-3
MAQRQPHSPNQTLISITNDTESSSSVVSNDNTNKGWSGDNSPGIEALCAIYITYAVIISVGILGNAILIKVFFKTKSMQTVPNIFITSLAFGDLLLLLTCVPVDATHYLAEGWLFGRIGCKVLSFIRLTSVGVSVFTLTILSADRYKAVVKPLERQPSNAILKTCVKAGCVWIVSMIFALPEAIFSNVYTFRDPNKNMTFESCTSYPVSKKLLQEIHSLLCFLVFYIIPLSIISVYYSLIARTLYKSTLNIPTEEQSHARKQIESRKRIARTVLVLVALFALCWLPNHLLYLYHSFTSQTYVDPSAMHFIFTIFSRVLAFSNSCVNPFALYWLSKSFQKHFKAQLFCCKAERPEPPVADTSLTTLAVMGTVPGTGSIQMSEISVTSFTGCSVKQAEDRF

SEQ ID NO: 5 - DNA, 28 bp, Artificial Sequence
  PCR primer
ggatcctgacaccacctcattactagac

SEQ ID NO: 6 - DNA, 24 bp, Artificial Sequence
  PCR primer
aagcttaaagctggatacacacag

SEQ ID NO: 7 - DNA, 44 bp, Artificial Sequence
  Probe
cacacagtgcttctattcctggagagttgtctccggtccatcct

SEQ ID NO: 8 - DNA, 45 bp, Artificial Sequence
  Probe
aggtagtgggttgcatccactggcacacaagtcagcaggagtaac

SEQ ID NO: 9 - DNA, 45 bp, Artificial Sequence
  Probe
tggcttcacgactgctttgtatctgtcagcgctgagaattgtcag

SEQ ID NO: 10 - protein, 9 aa, Artificial Sequence
  peptide HA epitope-tag
YPYDVPDYA

(19) United States
(12) Patent Application Publication
(10) Pub. No.: US 2005/0222402 A1
(43) Pub. Date: Oct. 6, 2005

(54) Dna encoding rat bombesin receptor subtype-3 (brs-3) and uses thereof

(76) Inventors: Jie Liu, West Chester, PA (US); Tung M. Fong, Somerset, NJ (US); Leonardus H.T. Van Der Ploeg, Scotch Plains, NJ (US)

Correspondence Address: MERCK AND CO., INC, P O BOX 2000, RAHWAY, NJ, 07065-0907, US

(21) Appl. No.: 10/486,414

(22) PCT Filed: Aug. 7, 2002
(86) PCT No.: PCT/US02/24971

§ 371 Date: Feb. 9, 2004

Related U.S. Application Data
(60) Provisional application No. 60/311,014, filed on Aug. 9, 2001.

(51) Int. Cl. C07K 14/705; C07H 21/04; C12N 15/09; C12N 5/06

(52) U.S. Cl. 536/23.5; 530/350; 435/69.1; 435/353; 435/320.1

(57) ABSTRACT

A rat bombesin receptor subtype-3 has been isolated, cloned and sequenced. This receptor is characteristic of the G-protein family of receptors. Isolation of rat bombesin receptor subtype-3 may be used to screen and identify novel bombesin receptor modulators that may contribute to the regulation of endocrine processes, metabolism, or the cell cycle. Such compounds may be used in the treatment of conditions that result from deregulated expression of bombesin receptor subtype-3.

### CROSS-REFERENCE TO RELATED APPLICATIONS

Not applicable.

### STATEMENT REGARDING FEDERALLY-SPONSORED R&D

Not applicable.

### REFERENCE TO MICROFICHE APPENDIX

Not applicable.

### FIELD OF THE INVENTION

The present invention relates to rat bombesin receptor subtype-3, designated rBRS-3, to isolated nucleic acid molecules which encode this receptor, to recombinant vectors and hosts comprising DNA encoding this receptor and to use of rBRS-3 in various assays.

### BACKGROUND OF THE INVENTION

Bombesin, a tetradecapeptide originally isolated from frog skin, represents the first member of a large family of regulatory peptides named bombesin-like peptides. Two bombesin-like peptides, gastrin-releasing peptide (GRP) (McDonald et al., Biochem. Biophys. Res. Commun. 90: 227-33 (1979)) and neuromedin B (NMB) (Minamino et al., Biochem. Biophys. Res. Commun. 114: 541-548 (1983)) have been found in mammals.

The orphan receptor bombesin receptor subtype-3 (BRS-3, also named BB3) is one of three subtypes of bombesin receptors, which was identified based on its high degree of homology to mammalian bombesin receptors. BRS-3 is a member of the G protein-coupled receptor superfamily and has been cloned from human, mouse and sheep (Whitley et al., J. Mol. Endocrinol. 23: 107-16 (1999)). A naturally occurring high affinity ligand for BRS-3 has not been identified. However, a synthetic peptide, [D-Tyr6-betaAla11-Phe13-Nle14] bombesin (6-14) (hereinafter dYB, comprising SEQ ID NO:10) was shown to have high affinity for all three human bombesin receptor subtypes (Pradhan et al., Eur. J. Pharmacol. 343: 275-87 (1998)). However, the human receptor pharmacology does not always extend to other species homologs.

WO 01/10889 (published Feb. 15, 2001) discloses a molecule described as rat BRS-3. However, this receptor molecule appears not to be a full-length receptor as it is only 382 amino acids long.

Bombesin, bombesin-like peptides and related receptors participate in a diverse array of physiological processes. BRS-3 has been implicated in the regulation of neuroendocrine function and energy metabolism (Ohki et al. Nature 390: 165-69 (1997)). Mice lacking functional BRS-3 are hyperphagic and have a reduced metabolic rate, which leads to the development of obesity, hypertension and diabetes as adults. Additionally, bombesin-like peptides may contribute to the pathogenesis of some human carcinomas (For review, see Lebacq-Verheyden et al., in Handbook of Experimental Pharmacology, Sporn, M. N. and Roberts, A. B., eds., Vol. 95, pp. 71-124, Springer-Verlag, Berlin).

Despite the identification of the cDNA clones encoding bombesin receptor subtypes mentioned above, it would be advantageous to identify additional mammalian genes encoding bombesin receptor subtypes in order to allow screening to identify novel bombesin receptor modulators that may contribute to the regulation of endocrine processes, metabolism, or the cell cycle.

### SUMMARY OF THE INVENTION

The present invention relates to an isolated or purified nucleic acid molecule (polynucleotide) which encodes a novel rat bombesin receptor subtype-3 (hereinafter rBRS-3). The DNA molecules disclosed herein may be transfected into a host cell of choice wherein the recombinant host cell provides a source for substantial levels of an expressed functional rBRS-3 protein (SEQ ID NO:2). This receptor protein provides a screening target to identify modulators of bombesin and bombesin-like peptides, which may be involved in the pathogenesis of a variety of human disorders when deregulated.

The present invention also relates to isolated nucleic acid molecules comprising a sequence of nucleotides that encode a rat BRS-3 protein as set forth in SEQ ID NO:2.

The present invention further relates to an isolated nucleic acid molecule which encodes mRNA which expresses a novel rat BRS-3 protein, this DNA molecule comprising the nucleotide sequence disclosed herein as SEQ ID NO:1.

The present invention also relates to biologically active fragments or mutants of SEQ ID NO:1, which encode mRNA expressing a novel rBRS-3 protein. Any such biologically active fragment and/or mutant will encode either a protein or protein fragment which at least substantially mimics the pharmacological properties of the rBRS-3 protein, including but not limited to the rBRS-3 protein as set forth in SEQ ID NO:2. Any such polynucleotide includes but is not necessarily limited to nucleotide substitutions, deletions, additions, amino-terminal truncations and carboxy-terminal truncations such that these mutations encode mRNA which express a functional rBRS-3 protein in a eukaryotic cell, such as Xenopus oocytes, so as to be useful for screening for agonists and/or antagonists of rat BRS-3 activity.

The present invention further relates to a process for expressing a rat BRS-3 protein in a recombinant host cell, comprising:
(a) introducing a vector comprising the nucleic acid of claim 1 into a suitable host cell; and,
(b) culturing the host cell under conditions which allow expression of said rat BRS-3 protein.

A preferred aspect of this portion of the present invention is disclosed in FIG. 1 (SEQ ID NO:1), which shows a DNA molecule encoding a novel rBRS-3 protein (SEQ ID NO:2).

The isolated nucleic acid molecules of the present invention may include a deoxyribonucleic acid molecule (DNA), such as genomic DNA and complementary DNA (cDNA), which may be single (coding or noncoding strand) or double stranded, as well as synthetic DNA, such as a synthesized, single stranded polynucleotide. The isolated nucleic acid molecule of the present invention may also include a ribonucleic acid molecule (RNA).

The present invention also relates to recombinant vectors and recombinant host cells, both prokaryotic and eukaryotic, which contain the nucleic acid molecules disclosed throughout this specification.

A preferred aspect of this portion of the present invention is a substantially purified form of a rat BRS-3 protein which consists of the amino acid sequence disclosed in FIG. 2 (SEQ ID NO:2).

Another preferred aspect of the present invention relates to a substantially purified, fully processed (including proteolytic processing, glycosylation and/or phosphorylation), mature BRS-3 protein obtained from a recombinant host cell containing a DNA expression vector comprising nucleotide sequence as set forth in SEQ ID NO:1, which expresses the rBRS-3 protein. It is especially preferred that the recombinant host cell be a eukaryotic host cell, such as a mammalian cell line, or Xenopus oocytes, as noted above.

Another preferred aspect of the present invention relates to a substantially purified membrane preparation, partially purified membrane preparation, or cell lysate which has been obtained from a recombinant host cell transformed or transfected with a DNA expression vector which comprises and appropriately expresses a complete open reading frame as set forth in SEQ ID NO:1, resulting in a functional form of rBRS-3. The subcellular membrane fractions and/or membrane-containing cell lysates from the recombinant host cells (both prokaryotic and eukaryotic as well as both stably and transiently transformed cells) contain the functional and processed proteins encoded by the nucleic acids of the present invention. This recombinant-based membrane preparation may comprise a rat BRS-3 protein and is essentially free from contaminating proteins, including but not limited to other rat source proteins. Therefore, a preferred aspect of the invention is a membrane preparation which contains a rat BRS-3 comprising the functional form of the full length BRS-3 protein as disclosed in FIG. 2 (SEQ ID NO:2). These subcellular membrane fractions will comprise either wild type and/or mutant variations which are biologically functional forms of rat BRS-3 at levels substantially above endogenous levels. Any such protein will be useful in various assays described throughout this specification to select for modulators of the rBRS-3 protein. A preferred eukaryotic host cell of choice to express the rBRS-3 molecules of the present invention is a mammalian cell line, or Xenopus oocytes.

The present invention also relates to biologically active fragments and/or mutants of a rat BRS-3 protein, comprising the amino acid sequence as set forth in SEQ ID NO:2, including but not necessarily limited to amino acid substitutions, deletions, additions, amino terminal truncations and carboxy-terminal truncations such that these mutations provide for proteins or protein fragments of diagnostic, therapeutic or prophylactic use and would be useful for screening for selective modulators, including but not limited to agonists and/or antagonists for rat bombesin and bombesin-like peptide receptor pharmacology.

A preferred aspect of the present invention is disclosed in FIG. 2 (SEQ ID NO:2), which indicates the amino acid sequence of the rat BRS-3 protein of the present invention. Characterization of this protein will allow for screening to identify novel bombesin receptor subtype-3 modulators that may have a role in the regulation of endocrine processes or metabolism. As noted above, heterologous expression of rat BRS-3 disclosed herein is contemplated at levels substantially above endogenous levels and will allow for the pharmacological analysis of compounds which may contribute to the pathogeneis of a variety of human disorders associated with deregulated BRS-3 expression. Heterologous cell lines expressing a functional rat BRS-3 (e.g., functional forms of SEQ ID NO: 2), can be used to establish functional or binding assays to identify novel BRS-3 modulators that may be useful in the development of therapeutics for human diseases associated with deregulated BRS-3 expression.

The rat BRS-3 receptor proteins of the present invention may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

The present invention also relates to rat BRS-3 fusion constructs, including but not limited to fusion constructs which express a portion of the rat BRS-3 protein linked to various markers, including but in no way limited to GFP (Green fluorescent protein), the MYC epitope, GST, and Fc. Any such fusion constructs may be expressed in the cell line of interest and used to screen for modulators of the rat BRS-3 protein disclosed herein.

The present invention relates to methods of expressing rat BRS-3 proteins and biological equivalents disclosed herein, assays employing these gene products, recombinant host cells which comprise DNA constructs which express these proteins, and compounds identified through these assays which act as agonists or antagonists of BRS-3 activity.

As used herein, “substantially free from other nucleic acids” means at least 90%, preferably 95%, more preferably 99%, and even more preferably 99.9%, free of other nucleic acids. As used interchangeably, the terms “substantially free from other nucleic acids,” “substantially purified,” “isolated nucleic acid” or “purified nucleic acid” also refer to DNA molecules which comprise a coding region for a rat BRS-3 protein that has been purified away from other cellular components. Thus, a rat BRS-3 DNA preparation that is substantially free from other nucleic acids will contain, as a percent of its total nucleic acid, no more than 10%, preferably no more than 5%, more preferably no more than 1%, and even more preferably no more than 0.1%, of non-rat BRS-3 nucleic acids. Whether a given rat BRS-3 DNA preparation is substantially free from other nucleic acids can be determined by such conventional techniques of assessing nucleic acid purity as, e.g., agarose gel electrophoresis combined with appropriate staining methods, e.g., ethidium bromide staining, or by sequencing.

As used herein, “substantially free from other proteins” or “substantially purified” means at least 90%, preferably 95%, more preferably 99%, and even more preferably 99.9%, free of other proteins. Thus, a rat BRS-3 protein preparation that is substantially free from other proteins will contain, as a percent of its total protein, no more than 10%, preferably no more than 5%, more preferably no more than 1%, and even more preferably no more than 0.1%, of non-rat BRS-3 proteins. Whether a given rat BRS-3 protein preparation is substantially free from other proteins can be determined by such conventional techniques of assessing protein purity as, e.g., sodium dodecyl sulfate polyacrylamide gel electrophoresis (SDS-PAGE) combined with appropriate detection methods, e.g., silver staining or immunoblotting. As used interchangeably, the terms “substantially free from other proteins” or “substantially purified”, or “isolated rat BRS-3 protein” or “purified rat BRS-3 protein” also refer to rat BRS-3 protein that has been isolated from a natural source. Use of the term “isolated” or “purified” indicates that rat BRS-3 protein has been removed from its normal cellular environment. Thus, an isolated rat BRS-3 protein may be in a cell-free solution or placed in a different cellular environment from that in which it occurs naturally. The term isolated does not imply that an isolated rat BRS-3 protein is the only protein present, but instead means that an isolated rat BRS-3 protein is substantially free of other proteins and non-amino acid material (e.g., nucleic acids, lipids, carbohydrates) naturally associated with the rat BRS-3 protein in vivo. Thus, a rat BRS-3 protein that is recombinantly expressed in a prokaryotic or eukaryotic cell and substantially purified from this host cell which does not naturally (i.e., without intervention) express this BRS-3 protein is of course “isolated rat BRS-3 protein” under any circumstances referred to herein. As noted above, a rat BRS-3 protein preparation that is an isolated or purified rat BRS-3 protein will be substantially free from other proteins and will contain, as a percent of its total protein, no more than 10%, preferably no more than 5%, more preferably no more than 1%, and even more preferably no more than 0.1%, of non-rat BRS-3 proteins.

As used herein, “a conservative amino acid substitution” refers to the replacement of one amino acid residue by another, chemically similar, amino acid residue. Examples of such conservative substitutions are: substitution of one hydrophobic residue (isoleucine, leucine, valine, or methionine) for another; substitution of one polar residue for another polar residue of the same charge (e.g., arginine for lysine; glutamic acid for aspartic acid).

As used herein, “rBRS” refers to a—rat bombesin receptor subtype-3—

As used herein, the term “mammalian” will refer to any mammal, including a human being.

### BRIEF DESCRIPTION OF THE DRAWINGS

FIG. 1 shows the nucleotide sequence of rat BRS-3 cDNA, as set forth in SEQ ID NO:1.

FIG. 2 shows the predicted amino acid sequence of rat BRS-3 protein, as set forth in SEQ ID NO:2.

FIGS. 3A and 3B show the nucleotide sequence of rat BRS-3 cDNA, as set forth in SEQ ID NO:1, and the corresponding predicted amino acid sequence, as set forth in SEQ ID NO:2. The seven transmembrane domains (TM 1-7) are underlined. The sequence upstream of TM-1 is an extracellular domain, while sequence downstream of TM-7 is an intracellular domain.

FIG. 4 shows the genomic organization and relative positions of restriction endonuclease sites within the rBRS-3 gene. Introns are indicated by a dotted line. The approximate scale used in the map is indicated in the upper right corner.

FIG. 5 shows the alignment of rat (SEQ ID NO:2), mouse (SEQ ID NO:3) and human BRS-3 (SEQ ID NO:4) predicted peptide sequences. The seven transmembrane domains are underlined and regions involved in species specificity are boxed and highlighted.

FIG. 6 shows Northern blot analysis of BRS-3 mRNA expression in different rat tissues using rat BRS-3 cDNA as a probe (see EXAMPLE 2).

FIG. 7 presents the functional activity of rat BRS-3 in transfected 293EBNA cells, measured by the stimulation of phosphatidylinositol hydrolysis by peptide agonists, GRP, NMB, bombesin and dYB. The values are mean±SE of triplicates. See EXAMPLE 8.

FIGS. 8A and 8B show the stimulation of calcium mobilization through activation of rat, human and chimeric BRS-3 receptors by dYB. Changes in Fluo-3 fluorescence serve as an indicator of intracellular calcium concentration and were measured by a FLIPR. The plotted data is a representative of 3-5 experiments. The results of multiple experiments are summarized in EXAMPLE 10. FIG. 8A shows a comparison of rat (black circles) and human (black squares) BRS-3 concentration-dependent activation. FIG. 8B shows the functional activities of chimeric BRS-3 receptors, RBB3-NTerm (white circles), RBB3-E2 (white triangles), and RBB3-E3 (white squares). See EXAMPLE 12.

FIG. 9 depicts the expression levels of HA-epitope tagged rat and human BRS-3 on the surface of transfected 293EBNA cells as measured by ELISA using a monoclonal antibody against HA-epitope. See EXAMPLE 11.

FIG. 10 shows the functional activity of dYB on the rat BRS-3 substitution mutants RBB3-SQT (white squares), RBB3-VD (white diamonds), RBB3-AMH (white circles) and RBB3-IF (white triangles). Also shown is the activity of RBB3 (black circles) and the chimeric receptor RBB3-E3 (black squares). Changes in Fluo-3 fluorescence serve as an indicator of intracellular calcium concentration and were measured by a FLIPR. The plotted data is a representative of 3 experiments. The results of multiple experiments are summarized in EXAMPLE 13 and Table 1.

### DETAILED DESCRIPTION OF THE INVENTION

The present invention relates to an isolated nucleic acid molecule (polynucleotide) which encodes a rat bombesin receptor subtype-3 protein. The nucleic acid molecules of the present invention are substantially free from other nucleic acids. For most cloning purposes, DNA is a preferred nucleic acid. This invention also relates to various functional domains of this receptor, such as the extracellular domain and the intracellular domain, and to hybrid molecules comprising at least one of these sequences.

The present invention relates to an isolated nucleic acid molecule (polynucleotide) which encodes mRNA which expresses a novel rat bombesin receptor subtype-3 protein, this DNA molecule comprising the nucleotide sequence disclosed herein as SEQ ID NO:1.

The rBRS-3 nucleic acid molecule of the present invention was identified through RT-PCR as described in detail in Example 1. The isolated cDNA clones, associated vectors, hosts, recombinant subcellular fractions and membranes, and the expressed and mature forms of rBRS-3 are important tools for drug discovery.

The present invention relates in part to transgenic animals, either an invertebrate (e.g., C. elegans) or vertebrate (e.g., mouse), for which the gene encoding rBRS-3 has been introduced into the germline of the animal. The purpose of this would be to inactivate, in the host, one or several endogenous BRS-3 and observe the biological effects. One such effect may well be an acquired resistance to drugs that are agonists (activators) of BRS-3. In the case of drugs with suspected—but unproven—method of action (MOA) via BRS-3, such BRS-3-harboring transgenic animals may be used to confirm such an effect. Expression of the newly introduced gene encoding rBRS-3 into the host can be constitutive or inducible, depending on the type of promoter used to drive its expression. Also depending on the type of promoter used, expression of rBRS-3 can be targeted to a given tissue(s) or it can be generalized.

In preferred embodiments of the invention, DNA is ligated into a vector, and introduced into suitable host cells to produce transformed cell lines that express the rat BRS-3 protein, or a fragment thereof. The resulting cell lines can then be produced in quantity for reproducible quantitative analysis of the effects of drugs on receptor function.

In other embodiments, mRNA may be produced by in vitro transcription of DNA encoding the invention peptide. This mRNA can then be injected into Xenopus oocytes where the RNA directs the synthesis of the rat BRS-3 protein. Alternatively, the invention-encoding DNA can be directly injected into oocytes for expression of a functional invention peptide. The transfected mammalian cells or injected oocytes may then be used in the methods of drug screening provided herein.

Therefore, the heterologous expression of the rat BRS-3 protein will allow the pharmacological analysis of compounds which may contribute to the regulation of the endocrine system, cell cycle or metabolism. Heterologous cell lines expressing these rBRS-3 proteins can be used to establish functional or binding assays to identify novel rBRS-3 modulators that may be useful in the development of novel human therapeutics for diseases related to deregulated bombesin receptor expression.

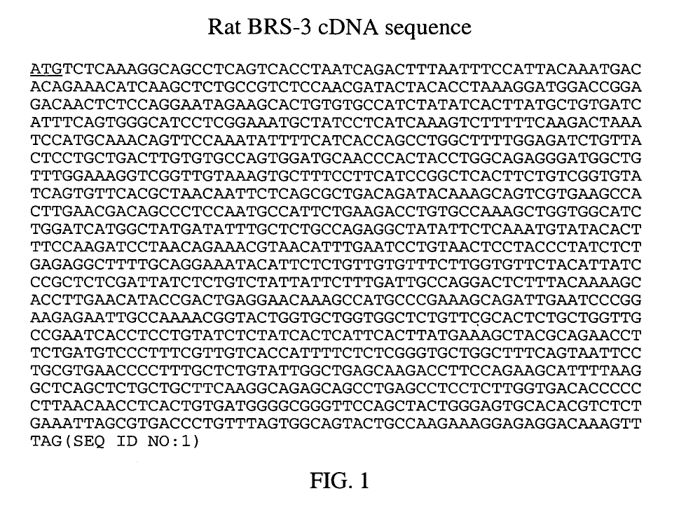

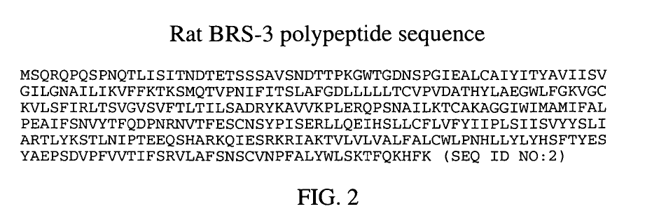

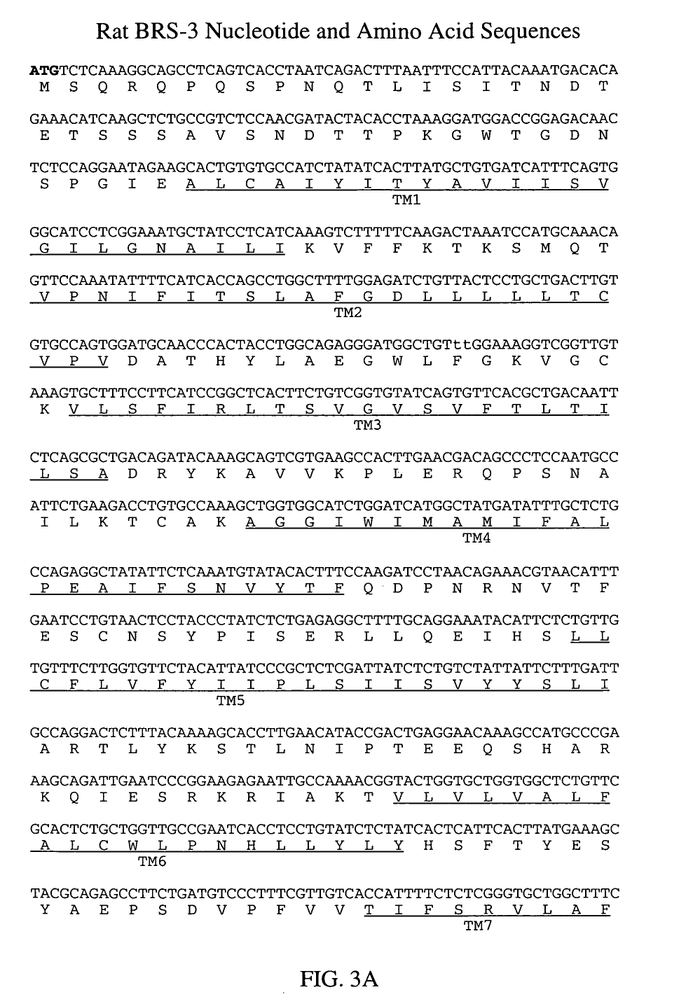

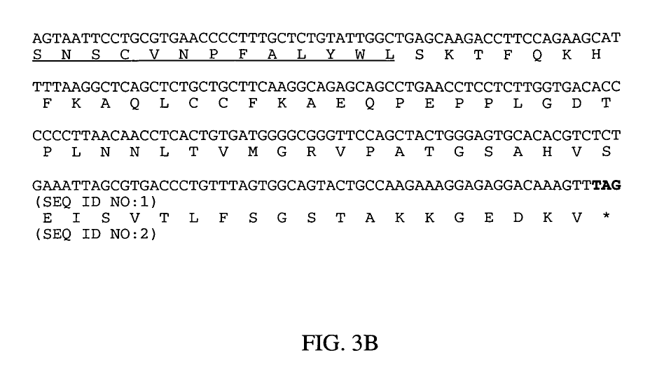

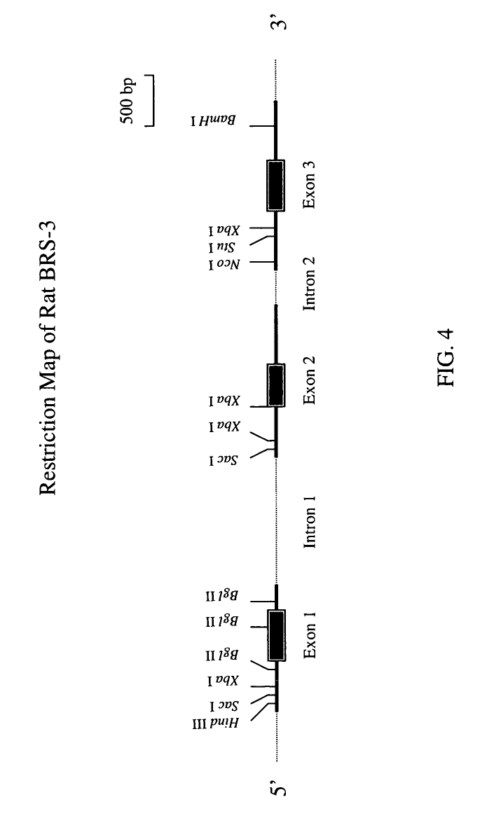

The present invention relates to the DNA molecule described in FIG. 1 as rat BRS-3 and set forth as SEQ ID NO:1, which encodes the rat bombesin receptor subtype-3 protein shown in FIG. 2 and set forth as SEQ ID NO:2.

This invention also relates to synthetic DNA that encodes the rBRS-3 protein where the nucleotide sequence of the synthetic DNA differs significantly from the nucleotide sequence of SEQ ID NO:1 but still encodes the same rBRS-3 protein as SEQ ID NO:2. Such synthetic DNAs are intended to be within the scope of the present invention. If it is desired to express such synthetic DNAs in a particular host cell or organism, the codon usage of such synthetic DNAs can be adjusted to reflect the codon usage of that particular host, thus leading to higher levels of expression of the BRS-3 protein in the host.

Therefore, the present invention discloses codon redundancy which may result in differing DNA molecules expressing an identical protein. For purposes of this specification, a sequence bearing one or more replaced codons will be defined as a degenerate variation. Also included within the scope of this invention are mutations either in the DNA sequence or the translated protein that do not substantially alter the ultimate physical properties of the expressed protein. For example, substitution of valine for leucine, arginine for lysine, or asparagine for glutamine may not cause a change in the functionality of the polypeptide.

It is known that DNA sequences coding for a peptide may be altered so as to code for a peptide that has properties that are different than those of the naturally occurring peptide. Methods of altering the DNA sequences include but are not limited to site directed mutagenesis. Examples of altered properties include but are not limited to changes in the affinity of an enzyme for a substrate or receptor for a ligand.

The present invention also relates to recombinant vectors and recombinant hosts, both prokaryotic and eukaryotic, which contain the substantially purified nucleic acid molecules disclosed throughout this specification. These vectors may be comprised of DNA or RNA; for most cloning purposes DNA vectors are preferred. Typical vectors include plasmids, modified viruses, bacteriophage, cosmids, yeast artificial chromosomes, and other forms of episomal or integrated DNA that can encode a rBRS-3 protein. It is well within the purview of the skilled artisan to determine an appropriate vector for a particular gene transfer or other use.

This invention also encompasses a method for identifying compounds that modulate rat bombesin receptor subtype-3 expression, comprising contacting a test compound with rat bombesin receptor subtype-3, and determining whether the test compound interacts with rat bombesin receptor subtype-3.

Included in the present invention are DNA sequences that hybridize to SEQ ID NO:1 under stringent conditions. By way of example, and not limitation, a procedure using conditions of high stringency is as follows: Prehybridization of filters containing DNA is carried out for 2 hours to overnight at 65° C. in buffer composed of 6×SSC, 5×Denhardt's solution, and 100 μg/ml denatured salmon sperm DNA. Filters are hybridized for 12 to 48 hrs at 65° C. in prehybridization mixture containing 100 μg/ml denatured salmon sperm DNA and 5-20×10⁶ cpm of ³²P-labeled probe. Washing of filters is done at 37° C. for 1 hr in a solution containing 2×SSC, 0.1% SDS. This is followed by a wash in 0.1×SSC, 0.1% SDS at 50° C. for 45 min. before autoradiography. Other procedures using conditions of high stringency would include either a hybridization step carried out in 5×SSC, 5×Denhardt's solution, 50% formamide at 42° C. for 12 to 48 hours or a washing step carried out in 0.2×SSPE, 0.2% SDS at 65° C. for 30 to 60 minutes.

Reagents mentioned in the foregoing procedures for carrying out high stringency hybridization are well known in the art. Details of the composition of these reagents can be found in, e.g., Sambrook et al., 1989, Molecular Cloning: A Laboratory Manual; Cold Spring Harbor Laboratory, Cold Spring Harbor, N.Y., which is hereby incorporated by reference. In addition to the foregoing, other conditions of high stringency which may be used are well known in the art.

Any of a variety of procedures may be used to clone rBRS-3. These methods include, but are not limited to, (1) a RACE PCR cloning technique (Frohman, et al., 1988, Proc. Natl. Acad. Sci. USA 85: 8998-9002). 5′ and/or 3′ RACE may be performed to generate a full-length cDNA sequence. This strategy involves using gene-specific oligonucleotide primers for PCR amplification of rBRS-3 cDNA. These gene-specific primers are designed through identification of an expressed sequence tag (EST) nucleotide sequence which has been identified by searching any number of publicly available nucleic acid and protein databases; (2) direct functional expression of the rBRS-3 cDNA following the construction of a rBRS-3-containing cDNA library in an appropriate expression vector system; (3) screening an rBRS-3-containing cDNA library constructed in a bacteriophage or plasmid shuttle vector with a labeled degenerate oligonucleotide probe designed from the amino acid sequence of the rBRS-3 protein; (4) screening an rBRS-3-containing cDNA library constructed in a bacteriophage or plasmid shuttle vector with a partial cDNA encoding the rBRS-3 protein. This partial cDNA is obtained by the specific PCR amplification of rBRS-3 DNA fragments through the design of degenerate oligonucleotide primers from the amino acid sequence known for other kinases which are related to the rBRS-3 protein; (5) screening a rBRS-3 -containing cDNA library constructed in a bacteriophage or plasmid shuttle vector with a partial cDNA or oligonucleotide with homology to a mammalian rBRS-3 protein. This strategy may also involve using gene-specific oligonucleotide primers for PCR amplification of rBRS-3 cDNA identified as an EST as described above; or (6) designing 5′ and 3′ gene specific oligonucleotides using SEQ ID NO:1 as a template so that either the full-length cDNA may be generated by known RACE techniques, or a portion of the coding region may be generated by these same known RACE techniques to generate and isolate a portion of the coding region to use as a probe to screen one of numerous types of cDNA and/or genomic libraries in order to isolate a full-length version of the nucleotide sequence encoding rBRS-3.

It is readily apparent to those skilled in the art that other types of libraries, as well as libraries constructed from other cell types-or species types, may be useful for isolating a rBRS-3-encoding DNA or a rBRS-3 homologue. Other types of libraries include, but are not limited to, cDNA libraries derived from other cells.

It is readily apparent to those skilled in the art that suitable cDNA libraries may be prepared from cells or cell lines which have rBRS-3 activity. The selection of cells or cell lines for use in preparing a cDNA library to isolate a cDNA encoding rBRS-3 may be done by first measuring cell-associated rBRS-3 activity using any known assay available for such a purpose.

Preparation of cDNA libraries can be performed by standard techniques well known in the art. Well known cDNA library construction techniques can be found for example, in Sambrook et al., 1989, Molecular Cloning: A Laboratory Manual; Cold Spring Harbor Laboratory, Cold Spring Harbor, N.Y. Complementary DNA libraries may also be obtained from numerous commercial sources, including but not limited to Clontech Laboratories, Inc. and Stratagene.

An expression vector containing DNA encoding a rBRS-3-like protein may be used for expression of rBRS-3 in a recombinant host cell. Such recombinant host cells can be cultured under suitable conditions to produce rBRS-3 or a biologically equivalent form. Expression vectors may include, but are not limited to, cloning vectors, modified cloning vectors, specifically designed plasmids or viruses. Commercially available mammalian expression vectors which may be suitable for recombinant rBRS-3 expression, include but are not limited to, pcDNA3.neo (Invitrogen), pcDNA3.1 (Invitrogen), pCI-neo (Promega), pLITMUS28, pLITMUS29, pLITMUS38 and pLITMUS39 (New England Bioloabs), pcDNAI, pcDNAIamp (Invitrogen), pcDNA3 (Invitrogen), pMC1neo (Stratagene), pXT1 (Stratagene), pSG5 (Stratagene), EBO-pSV2-neo (ATCC 37593) pBPV-1(8−2) (ATCC 37110), pdBPV-MMTneo(342−12) (ATCC 37224), pRSVgpt (ATCC 37199), pRSVneo (ATCC 37198), pSV2-dhfr (ATCC 37146), pUCTag (ATCC 37460), and IZD35 (ATCC 37565). Also, a variety of bacterial expression vectors may be used to express recombinant rBRS-3 in bacterial cells. Commercially available bacterial expression vectors which may be suitable for recombinant rBRS-3 expression include, but are not limited to pCR2.1 (Invitrogen), pET11a (Novagen), lambda gt11 (Invitrogen), and pKK223−3 (Pharmacia). In addition, a variety of fungal cell expression vectors may be used to express recombinant rBRS-3 in fungal cells. Commercially available fungal cell expression vectors which may be suitable for recombinant rBRS-3 expression include but are not limited to pYES2 (Invitrogen) and Pichia expression vector (Invitrogen). Also, a variety of insect cell expression vectors may be used to express recombinant protein in insect cells. Commercially available insect cell expression vectors which may be suitable for recombinant expression of rBRS-3 include but are not limited to pBlueBacIII and pBlueBacHis2 (Invitrogen), and pAcG2T (Pharmingen).

Recombinant host cells may be prokaryotic or eukaryotic, including but not limited to, bacteria such as E. coli, fungal cells such as yeast, mammalian cells including, but not limited to, cell lines of bovine, porcine, monkey and rodent origin; and insect cells including but not limited to Drosophila and silkworm derived cell lines. For instance, one insect expression system utilizes Spodoptera frugiperda (Sf21) insect cells (Invitrogen) in tandem with a baculovirus expression vector (pAcG2T, Pharmingen). Also, mammalian species which may be suitable and which are commercially available, include but are not limited to, L cells L-M(TK⁻) (ATCC CCL 1.3), L cells L-M (ATCC CCL 1.2), Saos-2 (ATCC HTB-85), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171) and CPAE (ATCC CCL 209).

As noted above in regard to the use of Xenopus oocytes to express a rBRS-3 gene of interest, the present invention is directed to methods for screening for compounds which modulate the expression of DNA or RNA encoding a rBRS-3 protein. Compounds which modulate these activities may be DNA, RNA, peptides, proteins, or non-proteinaceous organic molecules. Compounds may modulate by increasing or attenuating the expression of DNA or RNA encoding rBRS-3, or the function of the rBRS-3 protein. Compounds that modulate the expression of DNA or RNA encoding rBRS-3 or the biological function thereof may be detected by a variety of assays. The assay may be a simple “yes/no” assay to determine whether there is a change in expression or function. The assay may be made quantitative by comparing the expression or function of a test sample with the levels of expression or function in a standard sample. Kits containing rBRS-3, antibodies to rBRS-3, or modified rBRS-3 may be prepared by known methods for such uses.

The DNA molecules, RNA molecules, recombinant protein and antibodies of the present invention may be used to screen and measure levels of rBRS-3. The recombinant proteins, DNA molecules, RNA molecules and antibodies lend themselves to the formulation of kits suitable for the detection and typing of rBRS-3. Such a kit would comprise a compartmentalized carrier suitable to hold in close confinement at least one container. The carrier would further comprise reagents such as recombinant rBRS-3 or anti-rBRS-3 antibodies suitable for detecting rBRS-3. The carrier may also contain a means for detection such as labeled antigen or enzyme substrates or the like.

The assays described above can be carried out with cells that have been transiently or stably transfected with rBRS-3. The expression vector may be introduced into host cells via any one of a number of techniques including but not limited to transformation, transfection, protoplast fusion, and electroporation. Transfection is meant to include any method known in the art for introducing rBRS-3 into the test cells. For example, transfection includes calcium phosphate or calcium chloride mediated transfection, lipofection, infection with a retroviral construct containing rBRS-3, and electroporation. The expression vector-containing cells are individually analyzed to determine whether they produce rBRS-3 protein. Identification of rBRS-3 expressing cells may be done by several means, including but not limited to immunological reactivity with anti-rBRS-3 antibodies, and labeled ligand binding.

The specificity of binding of compounds showing affinity for rBRS-3 is shown by measuring the affinity of the compounds for recombinant cells expressing the cloned receptor or for membranes from these cells. Expression of the cloned receptor and screening for compounds that bind to rBRS-3 or that inhibit the binding of a radiolabeled ligand of rBRS-3 (such as the synthetic peptide, dYB to these cells, or membranes prepared from these cells, provides an effective method for the rapid selection of compounds with high affinity for rBRS-3. Such ligands need not necessarily be radiolabeled but can also be nonisotopic compounds that can be used to displace bound radiolabeled compounds or that can be used as activators in functional assays. Compounds identified by the above method are likely to be agonists or antagonists of rBRS-3 and may be peptides, proteins, or non-proteinaceous organic molecules.

Accordingly, the present invention is directed to methods for screening for compounds which modulate the expression of DNA or RNA encoding a rBRS-3 protein as well as compounds which effect the function of the rBRS-3 protein. Methods for identifying agonists and antagonists of other receptors are well known in the art and can be adapted to identify agonists and antagonists of rBRS-3. For example, Cascieri et al. (1992, Molec. Pharmacol. 41:1096-1099) describe a method for identifying substances that inhibit agonist binding to rat neurokinin receptors and thus are potential agonists or antagonists of neurokinin receptors. The method involves transfecting COS cells with expression vectors containing rat neurokinin receptors, allowing the transfected cells to grow for a time sufficient to allow the neurokinin receptors to be expressed, harvesting the transfected cells and resuspending the cells in assay buffer containing a known radioactively labeled agonist of the neurokinin receptors either in the presence or the absence of the substance, and then measuring the binding of the radioactively labeled known agonist of the neurokinin receptor to the neurokinin receptor. If the amount of binding of the known agonist is less in the presence of the substance than in the absence of the substance, then the substance is a potential agonist or antagonist of the neurokinin receptor. Where binding of the substance such as an agonist or antagonist to is measured, such binding can be measured by employing a labeled substance or agonist. The substance or agonist can be labeled in any convenient manner known to the art, e.g., radioactively, fluorescently, enzymatically.

Therefore, the specificity of binding of compounds having affinity for rBRS-3 shown by measuring the affinity of the compounds for recombinant cells expressing the cloned receptor or for membranes from these cells. Expression of the cloned receptor and screening for compounds that bind to rBRS-3 or that inhibit the binding of a known, radiolabeled ligand of rBRS-3 (such as the synthetic peptide, [D-Tyr-betaAla-Phe-Nle] bombesin) to these cells, or membranes prepared from these cells, provides an effective method for the rapid selection of compounds with high affinity for rBRS-3. Such ligands need not necessarily be radiolabeled but can also be nonisotopic compounds that can be used to displace bound radiolabeled compounds or that can be used as activators in functional assays. Compounds identified by the above method again are likely to be agonists or antagonists of rBRS-3 and may be peptides, proteins, or non-proteinaceous organic molecules. As noted elsewhere in this specification, compounds may modulate by increasing or attenuating the expression of DNA or RNA encoding rBRS-3, or by acting as an agonist or antagonist of the rBRS-3 protein. Again, these compounds that modulate the expression of DNA or RNA encoding rBRS-3 or the biological function thereof may be detected by a variety of assays. The assay may be a simple “yes/no” assay to determine whether there is a change in expression or function. The assay may be made quantitative by comparing the expression or function of a test sample with the levels of expression or function in a standard sample.

Expression of rBRS-3 DNA may also be performed using in vitro produced synthetic mRNA. Synthetic mRNA can be efficiently translated in various cell-free systems, including but not limited to wheat germ extracts and reticulocyte extracts, as well as efficiently translated in cell based systems, including but not limited to microinjection into frog oocytes, with microinjection into frog oocytes being preferred.

Following expression of rBRS-3 in a host cell, rBRS-3 protein may be recovered to provide rBRS-3 protein in active form. Several rBRS-3 protein purification procedures are available and suitable for use. Recombinant rBRS-3 protein may be purified from cell lysates and extracts by various combinations of, or individual application of salt fractionation, ion exchange chromatography, size exclusion chromatography, hydroxylapatite adsorption chromatography and hydrophobic interaction chromatography. In addition, recombinant rBRS-3 protein can be separated from other cellular proteins by use of an immunoaffinity column made with monoclonal or polyclonal antibodies specific for full-length rBRS-3 protein, or polypeptide fragments of rBRS-3 protein.

The present invention includes chimeric polypeptides wherein at least one domain of the rat BRS-3 polypeptide is linked a non-rat BRS-3 sequence of amino acid residues to produce a chimeric polypeptide.

In a particular embodiment of this invention, the chimeric polypeptide comprises an amino acid sequence of rat BRS-3 as set forth in residues 294-311 of SEQ ID NO: 2; linked with a non-rat BRS-3 amino acid sequence.

It is well understood in the art that differing DNA molecules may express an identical protein due to codon redundancy. Therefore, the present invention also includes isolated nucleic acid molecules, comprising a sequence of nucleotides that encodes said chimeric polypeptide.

In another embodiment of the present invention, the third extracellular loop (E3) of rat BRS-3 is switched with the E3 domain of human BRS-3 to produce a chimeric receptor, RB3-E3 (see EXAMPLE 12). Therefore, the present invention includes a chimeric polypeptide comprising a sequence of amino acid residues as set forth in SEQ ID NO:2, wherein a region defined by amino acid residues 294-311 is replaced by a region defined by amino acid residues 294-311 of SEQ ID NO:4.

Pharmaceutically useful compositions comprising modulators of rBRS-3 may be formulated according to known methods such as by the admixture of a pharmaceutically acceptable carrier. Examples of such carriers and methods of formulation may be found in Remington's Pharmaceutical Sciences. To form a pharmaceutically acceptable composition suitable for effective administration, such compositions will contain an effective amount of the protein, DNA, RNA, modified rBRS-3, or either rBRS-3 agonists or antagonists.

Rat and human BRS-3 have different tissue-specific expression patterns (see EXAMPLE 2) and different pharmacological properties. Surprisingly, rat BRS-3 has an approximately 1000-fold lower affinity to dYB than human BRS-3. Such drastic differences result from the variations in the amino acid sequence of the third extracellular loop of the receptor. As a result of these differences, rat BRS-3 will find use in identifying therapeutic compounds useful in proof of concept studies in rodents.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims.

### EXAMPLE 1

### Isolation and Characterization of DNA Encoding Rat Bombesin Receptor Subtype-3

Molecular procedures were performed following standard procedures well known in the art (See, e.g., Ausubel et. al. Short Protocols in Molecular Biology, F.M.,—2^{nd}. ed., John Wiley & Sons, (1992) and Sambrook et al., Molecular Cloning, A Laboratory Manual, 2^{nd} ed., Cold Spring Harbor Laboratory Press (1989), which are hereby incorporated by reference).

A cDNA clone specific to rat BRS-3 was generated by polymerase chain reaction (PCR) of rat brain hypothalamus cDNA. Oligonucleotide primers flanking the start (Forward: 5′-GGATCCTGACACCACCTCATTAC TAGAC-3′ (SEQ ID NO:5); 100 bp upstream of the start codon) and stop codons (Reverse: 5′-AAGCTTAAAGCTGGATACACACAG-3′ (SEQ ID NO:6); 60 bp downstream of the stop codon) of the published genomic DNA sequence of mouse BRS-3 (Weber et al., Gene 211(1), 125-3 (1998)) were designed. BamH I and Hind III sites were artificially introduced at the 5′-end of the primers for the convenience of subsequent cloning of the PCR product into an expression vector.

The forward and reverse primers above were utilized to PCR amplify full-length rat BRS-3 cDNA. The resulting 1.4 kb PCR product was cloned into the pE3 expression vector (Liu et al. J. Biomol Screen 3, 199-206 (1998), which is hereby incorporated by reference) for sequence analysis and functional characterization.

The rat BRS-3 gene was cloned by screening a genomic DNA library using the above-described rat BRS-3 cDNA as a probe. Approximately 1×10⁶ plaques from a Sprague-Dawley rat genomic library in Lambda FIX II vector (Stratagene, La Jolla, Calif.) were screened with ³²P-labeled probes. The filters were incubated overnight at 65° C. in ExpressHyb hybridization solution (Clontech, Palo Alto, Calif.) containing 5×10⁵ cpm/ml denatured probes. Filters were washed 3 times at 65° C. for 20 minutes in 2×SSC, 0.1% SDS. Seven plaques were purified, and two of them were found to contain all three coding exons and chosen for sequence analysis.

### EXAMPLE 2

### Northern Blot Analysis of rBRS-3 Expression

Tissue-specific expression of BRS-3 mRNA was studied by Northern blot analysis of an MTN blot (Clontech, Palo Alto, Calif.), which contains mRNA from eight different rat tissues. ³²P-labeled probes specific to the rat cDNA coding region were prepared by random primer extension using rat BRS-3 cDNA as the template. Highly stringent conditions were used for the hybridization and washing steps (Sambrook et al. Molecular Cloning: A Laboratory Manual, 2^{nd} Ed., Cold Spring Harbor Laboratory Press (1989), which is hereby incorporated by reference).

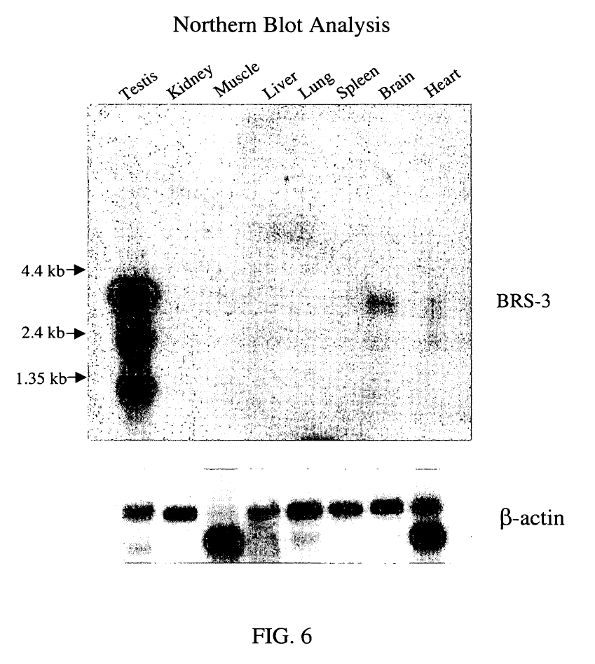

Abundant expression of an approximately 4 kb BRS-3 mRNA transcript was detected in rat testis. Additionally, weak BRS-3 expression was detected in rat brain and liver. No BRS-3 expression was detected in rat lung or kidney. (FIG. 6).

Tissue distribution of BRS-3 mRNA appears to be species-dependent. Rat testis expresses a high level of BRS-3 mRNA (see FIG. 6). Contrarily, BRS-3 expression was barely detectable in human and mouse testis and was undetectable in sheep testis (Whitley et al. J Mol Endocrinol 23(1): 107-16 (1999); Ohki-Hamazaki et al. Brain Res 762: 165-72 (1997); Fathi et al. J Biol Chem 268: 5979-84 (1993)). Additionally, BRS-3 expression is detectable in guinea pig and human uterus, but not in mouse or sheep uterus (Gorbulev et al. Eur J Biochem 208(2): 405-10 (1992)).

### EXAMPLE 3

### In Situ Hybridization Analysis of rBRS-3 Expression in Brain

Weak expression of BRS-3 mRNA has been reported in mouse and guinea pig brain. Similarly, weak expression of a BRS-3 transcript was detected in rat brain. However, the distribution pattern of BRS-3 mRNA in specific brain regions has not been well studied.

Therefore, in situ hybridization was carried out on coronal brain sections (14 um) of adult Sprague-Dawley rats. Using three gene-specific antisense 45 mer oligonucleotides, expression of BRS-3 mRNA in rat brain was mapped by in situ hybridization. Each of the oligonucleotides were labeled with [α-³³P]-dATP and used as probes for hybridization. Hybridization and washing conditions were as described (Guan and Van der Ploeg. Mol Brain Res 59: 273-279 (1998), which is hereby incorporated by reference). The nucleotide sequences of the three probes were as follows:

| | |
|---|---|
| Probe 1: | |
| 5′-CACACAGTGCTTCTATTCCTGGAGA | |
| GTTGTCTCCGGTCCATCCT-3′; | (SEQ ID NO:7) |
| Probe 2: | |
| 5′-AGGTAGTGGGTTGCATCCACTGGCA | |
| CACAAGTCAGCAGGAGTAAC-3′; | (SEQ ID NO:8) |
| Probe 3: | |
| 5′-TGGCTTCACGACTGCTTTGTATCTG | |
| TCAGCGCTGAGAATTGTCAG-3′. | (SEQ ID NO:9) |

Discrete expression was detected in habenula nuclei. Weak signals were also detected in the hypothalamus and striatum. BRS-3 expression was not detected in rat cortex, cerebellum and brain stem.

### EXAMPLE 4

### Construction of Chimeric and Substitution Mutant Receptors

Chimeric rat/human BRS-3 genes and substitution mutant rat BRS-3 genes were constructed by using PCR mutagenesis techniques (Fong, T. in Structure-function analysis of G protein coupled receptors, J, Wess, ed, Wiley-Liss, pp. 1-20, (1999)). All the mutant and wild type BRS-3 genes were cloned into pE3 expression vector which contains CMV early promoter, hygromycin-resistance gene and OriP segment from Epstein-Bar virus. The correctness of all mutant receptors was confirmed by sequencing analysis of the entire coding region.

### EXAMPLE 5

### Transfection and Expression

Wild type and mutant BRS-3 constructs were transfected by using lipofectamine reagents (Life Technologies, Rockville, Md.) into 293EBNA cells (Invitrogen, Carlsbad, Calif.) which support episomal replication of plasmid containing Epstein Bar virus OriP segment. Stable cell lines were obtained by selection with 250 ug/ml hygromycin. The cells were maintained as monolayers in Dulbecco's modified Eagle's medium (Life Technologies, Rockville, Md.) supplemented with 10% fetal bovine serum and incubated at 37° C. in a humidified 5% CO₂ incubator.

### EXAMPLE 6

### Membrane Preparation

Peptide dYB (dY-Q-W-A-V-(beta-A)-H-F-Nle-amide) (SEQ ID NO:10) was made by Research Genetics (Huntsville, Ala.) and custom labeled by NEN Life Science (NEN, Boston, Mass.) with a ¹²⁵I on the dY phenyl ring. The specific activity of ¹²⁵I-dYB is 2200 ci/mmol. The 293EBNA cells expressing wild type or mutant BRS-3 grown in 150-mm² dishes were harvested in binding buffer (50 mM HEPES, pH 7.4, 5 mM MgCl₂, 0.1% BSA) and disrupted with Dounce homogenization.

After removal of cell nuclei by a low speed centrifugation (1000×g) step, the cell membrane proteins were collected by centrifugation at 35,000×g for 20 minutes. The membrane protein concentration was determined by the Bradford method using BSA as standard.

### EXAMPLE 7

### Radioligand Binding Assay

For binding assays, ˜20 ug membrane proteins were incubated with 0.25 nM ¹²⁵I-dYB and various concentrations of competitors in 200 ul binding buffer. After a 2 hr incubation at room temperature, the binding reaction was terminated by filtering through a GF/c filter-mate and washing the filter with binding buffer using Packard 96-well Topcount Harvester. The amount of radioligand bound to the receptor was determined by measurement of the radioactivity on the filter through liquid scintillation counting. The non-specific binding was defined as the binding in the presence of 1 uM unlabeled dYB.

### EXAMPLE 8

### Phosphatidylinositol Hydrolysis Functional Assay

The functional properties of rat BRS-3 were characterized in transfected 293EBNA cells. The transfected 293EBNA cells were plated onto poly-D-lysine coated 12-well plates and incubated in the medium containing 2 uCi/ml [³H]myo-inositol. The cells were labeled for 48 hrs, and subsequently incubated for 30 minutes in Hank's balanced salt solution containing 10 mM HEPES, 0.1% BSA and 10 mM LiCl.

Since a natural agonist of BRS-3 has not been found, members of the bombesin-like peptide family, including GRP, NMB, bombesin and dYB, were used to stimulate the transfected cells described above, in the same buffer, for 1 hr at 37° C. The reaction was stopped by removal of medium and addition of 10 mM formic acid. The inositol monophosphate fraction was then isolated by anion chromatography as described (Berridge et al. Biochem. J. 212: 473-482 (1983), which is hereby incorporated by reference).

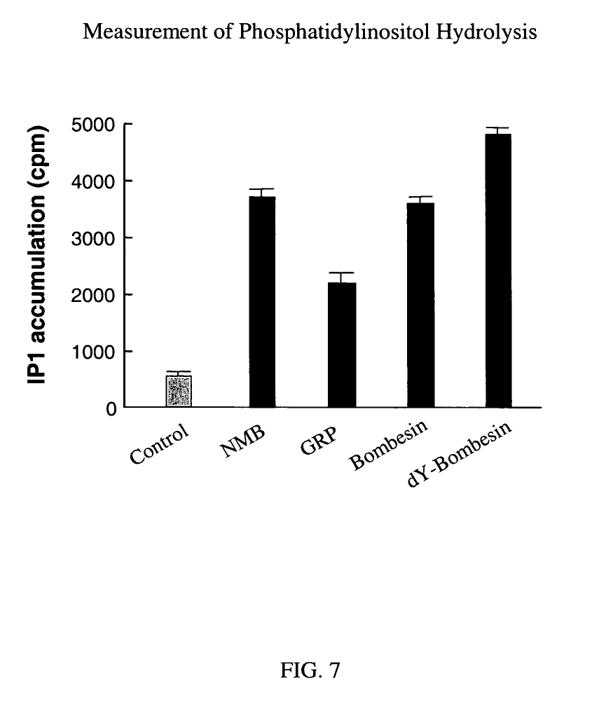

As expected, each of the four peptides at 10 uM stimulated a significant increase in phosphatidylinositol (PI) hydrolysis, as indicated by inositol monophosphate production in the cell (see FIG. 7). The most effective peptide was dYB, which stimulated a 9-fold increase in PI hydrolysis, followed by NMB, bombesin and GRP. As a control, non-transfected 293EBNA cells did not give any PI hydrolysis response to the four peptides tested (data not shown).

### EXAMPLE 9

### Calcium Mobilization Assay

The functional activity of rat BRS-3 was also studied by measuring the agonist-induced calcium mobilization response using a Fluorescence Imaging Plate Reader (Molecular Devices, Sunnyvale, Calif.). Transfected 293EBNA cells were plated onto poly-D-lysine coated 96-well plates with black wall and clear bottom (Becton Dickinson, Franklin Lakes, N.J.). After 16-48 hr culture, the cells were loaded with 4 uM Fluo-3-AM fluorescent calcium-indicator dye (Molecular Probes, Eugene, OR) in assay buffer (Hank's balanced salts solution, 10 mM HEPES, pH7.4, 0.1% bovine serum albumin, 2.5 mM probenecid).

The changes in intracellular calcium concentration, as indicated by Fluo-3 fluorescence, were immediately measured by FLIPR. Maximal changes in fluorescence over base line were used to determine agonist response.

A transient increase in intracellular calcium concentration was observed when the receptor was stimulated with 10 uM GRP, NMB, bombesin and dYB. This calcium response is believed to be downstream of inositol triphosphate (IP3) generation and result from release of calcium from intracellular storages. These peptides showed low potency at rat BRS-3. At concentrations below 1.0 uM, none of the four peptides could stimulate a significant calcium response.

### EXAMPLE 10

### Activation of BRS-3 by dYB

The synthetic peptide dYB was reported to have a nanomolar high affinity and high potency to all three human bombesin receptor subtypes, including human BRS-3 (Pradhan et al. Eur J Pharmacol 343: 275-87 (1998); Ryan et al. J Biol Chem 273: 13613-24 (1998)). However, specific binding or potent activation of rat BRS-3 by dYB was undetectable.

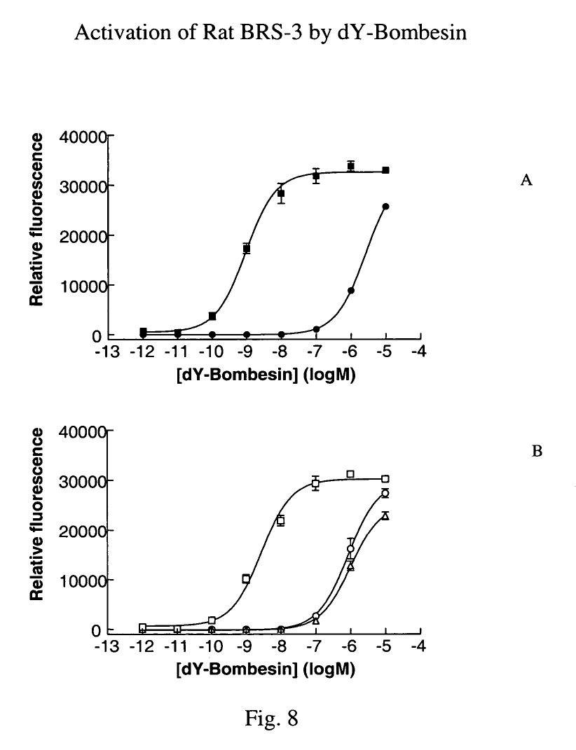

To determine if this discrepancy was due to species-dependent variation, rat BRS-3 was compared to its human and mouse homologs in the same expression system. An approximately 1000-fold difference in dYB potency between human and rodent BRS-3 was detected. As shown in FIG. 8A, dYB potently activated human BRS-3 with an EC50 value of 1.2±0.4 nM, whereas it only activated rat BRS-3 with an EC50 value of 2.2±0.5 uM. Similar to rat BRS-3, dYB also poorly activated mouse BRS-3, as indicated by an EC50 that was in the uM range (data not shown).

### EXAMPLE 11

### ELISA Assay

The observed difference in dYB potency may be due to a lower level of expression of rat BRS-3 as compared to that of human homolog. To test this theory, a nine-amino acid epitope (YPYDVPDYA; SEQ ID NO:11) derived from the influenza hemagglutinin protein (HA-tag) was added to the N-terminus of both the rat and human BRS-3 receptors. This allowed the amount of receptors present on the cell surface to be measured using a monoclonal antibody against the HA-epitope.

An indirect cellular ELISA approach was used to quantify the relative amount of tagged receptors on the cell surface (Schoneberg and Wess, J. Biol. Chem. 270(30): 18000-18006 (1995), which is hereby incorporated by reference). The 293EBNA cells transfected with tagged receptors were plated onto a 96-well plate. After overnight culture, the cells were fixed with 4% formaldehyde in PBS, and incubated with a monoclonal antibody directed against the HA-epitope tag (12CA5, Boehringer Mannheim). The plate was then washed and incubated with a horseradish peroxidase-conjugated goat anti-mouse IgG antibody (Amersham). The peroxidase activities present on the cell surface were measured by using TMB (Pierce, Rockford, Ill.) and H₂O₂ as substrates and chromogen.

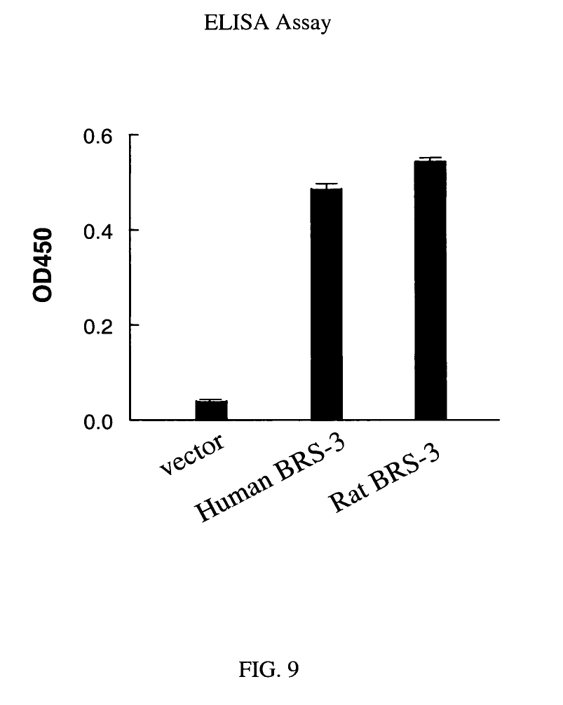

As shown in FIG. 9, rat BRS-3 was expressed at a high level on the surface of transfected 293EBNA cells. Similarly, human BRS-3 was also expressed at a high level. The difference in dYB potency was also observed in tagged receptors. These data rule out the possibility that a difference in the density of receptors is responsible for the observed dYB potency difference between rat and human.

### EXAMPLE 12

### Characterization of Species-Specificity by Chimeric Receptor Construction

It has been demonstrated extensively that the extracellular domains and the outer portion of the transmembrane domains of G protein-coupled receptors are directly involved in ligand binding and induction of receptor conformational changes that trigger down-stream signaling cascade (Wess, J. Structure-Function analysis of G Protein-Coupled Receptors, John Wiley, New York (1999)).

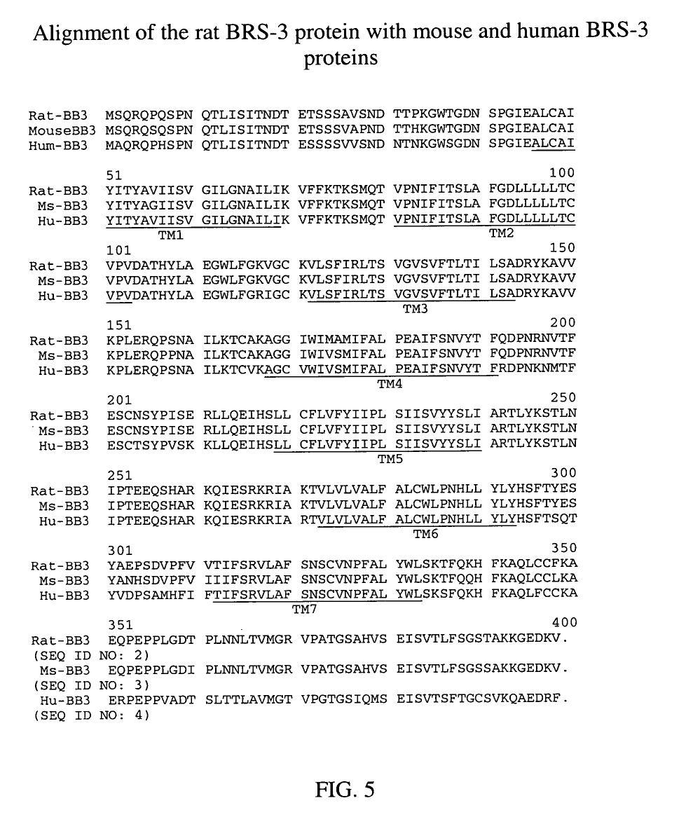

Alignment of rat, mouse and human BRS-3 peptide sequences shows that the variations in amino acids between rodent and human BRS-3 are mostly located in N- and C-terminal tails, E2- and E3 loops (FIG. 5). The C-terminal tail is very unlikely to participate in ligand binding. The species-dependent pharmacological difference exhibited by BRS-3 is, therefore, likely to be dictated by the differences in the three extracellular domains.

To determine the structural basis of the marked difference in dYB potencies between rat and human BRS-3, we constructed three chimeric receptor mutants in which the N-terminal-, E2-, or E3 loop of rat BRS-3 was replaced with corresponding human BRS-3 sequence (EXAMPLE 4). These chimeric receptors, RBB3-Nterm, RBB3-E2 and RBB3-E3, along with wild type rat and human BRS-3 were transfected into 293EBNA cells and functionally characterized by calcium mobilization assay using a FLIPR.

The results of said functional assays utilizing dYB at wild type and mutant BRS-3 receptors are shown in TABLE 1 below. The EC50 values were mean±S.E. calculated from dose-response curves of 3-5 repeated experiments using FLIPR assays.

TABLE 1

| Receptors | Abbreviation | Mutations | EC50, nM |
|---|---|---|---|
| Rat BRS-3 | RB3 | / | 2200 ± 550 |
| Human | HB3 | / | 1.2 ± 0.4 |
| BRS-3 | | | |
| Rat | RB3-E2 | switch 2^{nd} extracellular loop | 990 ± 140 |
| BRS-3/E2 | | | |
| Rat | RB3-E3 | switch 3^{rd} extracellular loop | 2.8 ± 0.6 |
| BRS-3/E3 | | | |
| Rat | RB3-SQT | Y²⁹⁸E²⁹⁹S³⁰⁰-S²⁹⁸Q²⁹⁹T³⁰⁰ | 390 ± 80 |
| BRS-3/SQT | | | |
| Rat | RB3-VD | A³⁰²E³⁰³-V³⁰²D³⁰³ | 930 ± 150 |
| BRS-3/VD | | | |
| Rat | RB3-AMH | D³⁰⁶V³⁰⁷P³⁰⁸-A³⁰⁶M³⁰⁷H³⁰⁸ | 370 ± 70 |
| BRS-3/AMH | | | |
| Rat | RB3-IF | V³¹⁰V³¹¹-I³¹⁰F³¹¹ | 1300 ± 230 |
| BRS-3/IF | | | |

Switching the N-terminal, and second extracellular loop did not significantly change the receptor property (FIG. 8B). However, switching the third extracellular loop (E3) of rat BRS-3 resulted in a chimeric receptor, RB3-E3, that behaved almost identically to human BRS-3. RB3-E3 could be activated by dYB with a high potency (EC50=2.8±0.6 nM) which was close to that of human BRS-3 (EC50=1.2±0.4 nM) (see Table 1).

The binding activities of the chimeric BRS-3 receptors were also evaluated using [¹²⁵I]dYB. Similar to wild type rat BRS-3, RBB3-Nterm and RBB3-E2 did not exhibit detectable specific binding signal at concentrations of 0.1 to 2 nM in membrane and whole cell binding assays. In contrast, the chimeric receptor RBB3-E3 showed a significant binding to [¹²⁵I]dYB which could be competitively displaced by unlabeled dYB with a high affinity (Ki=1.2±0.7 nM) which was almost identical to human BRS-3 (Ki=1.8±0.6 nM). Similar to human BRS-3, RBB3-E3 bound GRP, NMB, bombesin and phyllolitorin at uM low affinities, FIG. 8.

### EXAMPLE 13

### Characterization of Species-Specificity by Construction of Substitution Mutants

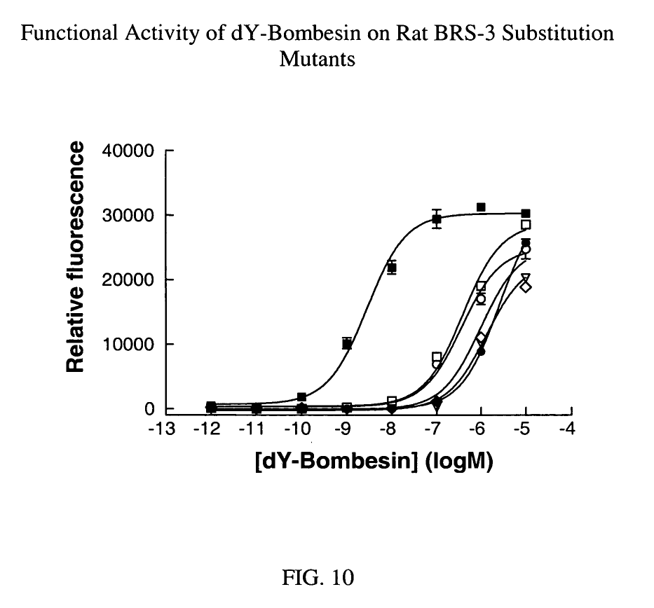

Among the 17 amino acids in E3 loop, 10 of them are conserved in rodents but different in human BRS-3 (FIG. 5). To determine which amino acids in the E3 loop of human BRS-3 conferred dYB activity upon rat BRS-3, several substitution BRS-3 mutants were made in which the amino acids in the E3 loop of rat BRS-3 were substituted with residues that match human BRS-3. As shown in FIG. 10 and Table 1, mutation of Y²⁹⁸E²⁹⁹S³⁰⁰ to S²⁹⁸Q²⁹⁹T³⁰⁰ (RB3-SQT) and D³⁰⁶V³⁰⁷p³⁰⁸ to A³⁰⁶M³⁰⁷H³⁰⁸ (RB3-AMH) partially mimicked the effect of switching entire E3 loop, the potency of dYB in stimulation of the receptor-mediated calcium mobilization was improved by 5- and 6-fold, respectively. On the other hand, mutations of A³⁰²E³⁰³ to V³⁰²D³⁰³ (RBB3-VD) and V³¹⁰V³¹¹ to I³¹⁰F³¹¹ (RBB3-IF) had little effect on the receptor activity. These results indicate that substitution of multiple residues in the E3 loop is probably required to gain high affinity dYB binding activity. It is also possible that the correct conformation of E3 loop is critically important for dYB binding. Overall, the E3 loop of BRS-3 is very likely to be involved in the interactions with dYB and possibly its native ligand as well. Consistent with this notion, residues in the E3 loops of NMB and GRP receptors were found to be critical for high affinity binding of their ligands NMB and GRP, respectively (Sainz et al. J. Biol. Chem. 273, 15927-15932 (1998); Akeson et al. J. Biol. Chem. 272, 17405-09 (1997)).

1. An isolated nucleic acid molecule, comprising a sequence of nucleotides that encodes a rat bombesin receptor subtype-3 (BRS-3) protein as set forth in SEQ ID NO:2.

2. The isolated nucleic acid molecule of claim 1 wherein the nucleic acid is DNA.

3. The isolated nucleic acid molecule of claim 1 wherein the nucleic acid is mRNA.

4. The isolated nucleic acid molecule of claim 1 wherein the nucleic acid is cDNA.

5. The isolated nucleic acid molecule of claim 1 wherein the sequence of nucleotides comprises the sequence of nucleotides set forth in SEQ ID NO:1.

6. A vector comprising the nucleic acid molecule of claim 1.

7. A host cell comprising the vector of claim 6.

8. A process for expressing a rat BRS-3 protein in a recombinant host cell, comprising:
(a) introducing a vector comprising the nucleic acid of claim 1 into a suitable host cell; and,
(b) culturing the host cell under conditions which allow expression of said rat BRS-3 protein.

9. (canceled)

10. An isolated and purified rat bombesin receptor subtype-3 (BRS-3) polypeptide comprising a sequence of amino acids as set forth in SEQ ID NO:2.

11. A method for identifying compounds that modulate rat bombesin receptor subtype-3 expression, comprising contacting a test compound with the rat BRS-3 polypeptide of claim 10 and determining whether the test compound interacts with rat bombesin receptor subtype-3.

12. The polypeptide of claim 10 wherein the amino acids Y²⁹⁸E²⁹⁹S³⁰⁰ are replaced with the amino acids S²⁹⁸Q²⁹⁹T³⁰⁰.

13. The polypeptide of claim 10 wherein the amino acids D³⁰⁶V³⁰⁷P³⁰⁸ are replaced with the amino acids A³⁰⁶M³⁰⁷H³⁰⁸.

14. A chimeric receptor comprising a sequence of amino acid residues as set forth in SEQ ID NO:2, wherein a region defined by amino acid residues 294-311 is replaced by a region defined by amino acid residues 294-311 of SEQ ID NO:4.

15. A chimeric polypeptide comprising an amino acid sequence of rat BRS-3 as set forth in residues 294-311 of SEQ ID NO:2; linked with a non-rat BRS-3 amino acid sequence.

16. An isolated nucleic acid molecule, comprising a sequence of nucleotides that encodes the chimeric polypeptide of claim 15.
